(19) United States
(12) Patent Application Publication
(10) Pub. No.: US 2024/0033095 A1
(43) Pub. Date: Feb. 1, 2024

(54) Systems and Methods to Account for Bone Quality to Reduce Stress Shielding in Implants

(71) Applicant: Mayo Foundation for Medical Education and Research, Rochester, MN (US)

(72) Inventor: John W. Sperling, Rochester, MN (US)

(21) Appl. No.: 18/255,647

(22) PCT Filed: Jul. 1, 2021
(86) PCT No.: PCT/US2021/040156

§ 371 Date: Jun. 2, 2023

Related U.S. Application Data
(60) Provisional application No. 63/120,930, filed on Dec. 3, 2020.

(51) Int. Cl. A61F 2/30; A61F 2/40; A61B 5/00

(52) U.S. Cl. CPC A61F 2/3094; A61F 2/4003; A61B 5/4509; A61F 2002/30011; A61F 2002/30014; A61F 2002/30069; A61F 2002/30324; A61F 2002/30948

(57) ABSTRACT

Systems and methods are provided for implant design and manufacturing to address stress shielding and/or implant fixation. The implant design and methodology may include accounting for the anatomy and quality of a bone of a subject to address stress shielding and/or implant fixation considerations for the subject. Implants or components may be asymmetrically designed to better match the associated anatomy as well as decrease stress shielding or improve implant fixation, such as by quantifying bone quality and matching properties of the implant or coatings of the implant to optimize engagement between the implant and the highest quality bone. Information derived from the methodology can be used to guide the design of the implant resulting in an asymmetric design that minimizes or eliminates stress shielding or improves implant fixation.

### CROSS-REFERENCE TO RELATED APPLICATIONS

This application claims the benefit of U.S. Provisional Patent Application Ser. No. 63/120,930 filed on Dec. 3, 2020 and entitled “Systems and Methods to Target Bone Quality to Reduce Stress Shielding in Implants,” which is incorporated herein by reference as if set forth in its entirety for all purposes.

### BACKGROUND

There has been a dramatic increase in the use of orthopedic and dental implants worldwide. Studies across anatomic areas have demonstrated that one of the central problems is the persistent and significant rate of stress shielding and associated bone loss.

Stress shielding refers to the reduction in bone density (osteopenia) as a result of removal of typical stress from bone by an implant (for instance, the stem of a joint prosthesis). This is because by Wolff s law, bone in a healthy person or animal will remodel in response to the loads it is placed under. After surgery, the bone shares its load with the implant. However, the modulus of elasticity varies between the metal and bone, resulting in changes of how the forces are distributed. Therefore, as the loading on a bone decreases, the bone will become less dense and weaker with resultant bone resorption. The magnitude of stress shielding and the specific locations of bone loss are based on the differences between the properties of the implant and the properties of the underlying bone.

There is extensive literature reporting that stress shielding can result in severe bone resorption across anatomic sites. For example, in shoulder replacement, short stem uncemented humeral components have been reported to have rates of bone resorption of greater than 40 to 70% in short term follow-up. In longer term studies, at a mean of 8 years, one reference reported stress shielding in 47% uncemented stems with partial or complete greater tuberosity resorption in 100% uncemented stems.

Stress shielding also occurs when plates or intramedullary nails are used to repair fractures. While the rigid nature of plates and nails helps to stabilize the fracture and facilitates early mobility, the increased stiffness of the plate or intramedullary nail results in bone loss due to decreased loading of the bone. It has been shown that bone remodeling is extremely sensitive to even small changes in cyclic bone stresses. Changes in cyclic bone stresses of even less than 1% of the ultimate strength can result in measurable changes in bone remodeling after only a few months.

Orthopedic and dental implants function as rigid osseous anchors within bone. The mechanical behavior of implant materials, surface coatings, and shape result in adaptive bone remodeling. The mechanical mismatch between host bone and metallic implants has been a long-lasting concern. For example, the elastic modulus of bone is 10-30 GPa. For two of the most commonly used implant materials, the elastic modulus is approximately 100 GPa for pure titanium and 230 GPa for cobalt-chrome. In this environment, contacted bone is often inappropriately stress shielded, and hence, implants lose supportive bone at the implant-bone region over time.

The current practice in orthopedics has been the use of implants that have been designed without regard to the variability of internal bone architecture. For example, the design and manufacturing of stems for joint arthroplasty has been traditionally driven by ease of and cost of manufacturing. Therefore, humeral stems have been designed to be symmetric and can be used in right or left sides. This minimizes inventory as well as the cost of manufacturing. This long held practice has been forcing the anatomy to fit the implant rather than making the implant match the anatomy and underlying bone architecture.

This concerning finding highlights the need to address the cause of stress shielding and the resultant need for technology and strategies to address this significant reason for implant failure.

### SUMMARY OF THE DISCLOSURE

The present disclosure addresses the aforementioned drawbacks by providing systems and methods for implant design and manufacturing to address stress shielding and/or to improve implant fixation to a bone. The implant design and methodology may include taking into account the anatomy and quality of a bone of a subject to address the stress shielding and fixation considerations for the subject. The implant design may be asymmetric, and/or may include asymmetric bone-growth promoting coatings applied at locations determined to mitigate stress shielding.

In one configuration, a method is provided for manufacturing an orthopedic implant for repairing a part of a bone in a subject. The method includes forming the implant to include at least one material property determined by: i) obtaining an image of the bone from at least one viewing plane; ii) orienting on the image a cross section of the bone; iii) quantifying a quality of the bone based on the cross section; and iv) determining the at least one material property of the implant to reduce stress shielding for the bone using the quantified bone quality.

In one configuration, a device is provided for repairing a part of a bone in a subject. The device includes a first section having a first material property and a second section having a second material property. The first section is connected to the second section at a central region of the device forming an asymmetric implant. The first section and the second section are configured to reduce stress shielding to a region of the bone.

In one configuration, a method is provided for manufacturing an orthopedic implant for repairing a part of a bone in a subject. The method includes forming the implant to include at least one material property determined by: i) obtaining an image of the bone from at least one viewing plane; ii) orienting on the image a cross section of the bone; iii) quantifying a quality of the bone based on the cross section; and iv) determining the at least one material property of the implant to improve implant fixation to the bone using the quantified bone quality.

In one configuration, a device is provided for repairing a part of a bone in a subject. The device includes a first section having a first material property and a second section having a second material property. The first section is connected to the second section at a central region of the device forming an asymmetric implant. The first section and the second section are configured to improve implant fixation to the bone.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention. Like reference numerals will be used to refer to like parts from Figure to Figure in the following description.

### BRIEF DESCRIPTION OF THE DRAWINGS

FIG. 1A is a cross section of a face of a prior art symmetric implant.

FIG. 1B is a flowchart of non-limiting example steps for configuring an implant or a material property of an implant to address stress shielding.

FIG. 1C is a flowchart of non-limiting example steps for configuring an implant or a material property of an implant to improve implant fixation.

FIG. 1D is a flowchart of non-limiting example steps for configuring an implant or a material property of an implant to address stress shielding and improve implant fixation.

FIG. 2 shows a traced computed tomography (CT) two-dimensional (2D) CT slice in a coronal viewing plane of the humerus with measurement lines shown in dashed lines.

FIG. 2A shows a traced computed tomography (CT) two-dimensional (2D) CT slice in a coronal viewing plane of the humerus.

FIG. 2B shows a traced computed tomography (CT) two-dimensional (2D) CT slice in a sagittal viewing plane of the humerus with measurement lines shown in dashed lines.

FIG. 2C shows a traced computed tomography (CT) two-dimensional (2D) CT slice in a sagittal viewing plane of the humerus.

FIG. 2D shows an axial cross section of a humerus with a depiction of the cross section's location on an image of a humerus bone in FIG. 2E with measurement lines shown.

FIG. 2E is an image of a humerus bone with the location of the cross section from FIG. 2D shown.

FIG. 3A is a non-limiting example cross section of a proximal humerus for bone quality assessment.

FIG. 3B is the non-limiting example for bone quality assessment of a proximal humerus of FIG. 3A shown at the level of a humeral cut.

FIG. 4 is a non-limiting example of an asymmetric coating implant.

FIG. 5 is a non-limiting example of an asymmetric fin length implant.

FIG. 6 is a non-limiting example of an asymmetric fin length implant with asymmetric coating.

FIG. 7 is a non-limiting example of an asymmetric fin thickness implant.

FIG. 8 is a non-limiting example of an asymmetric fin thickness with asymmetric coating thickness implant.

FIG. 9 is a non-limiting example of an asymmetric fin thickness with asymmetric fin length implant.

FIG. 10 is a non-limiting example of an asymmetric fin thickness with asymmetric fin length and asymmetric coating thickness implant.

FIG. 11 is a non-limiting example inlay implant implanted in a humerus that may be configured in accordance with the present disclosure.

FIG. 12 is a non-limiting example onlay implant implanted in a humerus that may be configured in accordance with the present disclosure.

FIG. 13 is a bar graph of non-limiting example results from a simulation protocol for the volume-weighted change in stress plotted by implant configuration.

FIG. 14 is a bar graph of non-limiting example results from a torque at failure test for each of three different implant models.

### DETAILED DESCRIPTION

Systems and methods are provided for implant design and manufacturing to address stress shielding. The implant design and methodology may include taking into account the anatomy and quality of a bone of a subject to address stress shielding considerations for the subject. Implants, components, or coatings can be asymmetrically designed to better match the associated anatomy as well as decrease stress shielding, such as by quantifying bone quality and matching material properties of the implant. Information derived from the methodology can be used to guide the design of the implant resulting in an asymmetric design and/or coatings that minimize or eliminates stress shielding. In some configurations, a method for implant design to minimize stress shielding includes an asymmetrical shape of the implant; an asymmetric coating applied to the implant, an asymmetric type of coating applied to the implant; an asymmetric modulus of elasticity of the implant material, and the like. Implants may be configured for repairing a part of a bone in a subject, such as by repairing a fracture, or through arthroplasty, and the like. Implant designs may include a variety of implant configurations, such as stemmed implants and stemless implants including inlay, onlay, or a hybrid approach.

In a non-limiting example, the methodology facilitates differentially engaging the better quality inferior and anterior humeral bone as well as the peripheral humeral bone for both immediate fixation as well as to prevent stress shielding. Adjusting the thickness of the fins can be done in several ways, including variable fin length, variable fin thickness, variable bone in-growth coating thickness on the surface of the implant, and the like. One will appreciate that fins can be any protrusion from an implant such as arms, blades, wings, and the like, or part of an implant, or a modified geometry of the implant.

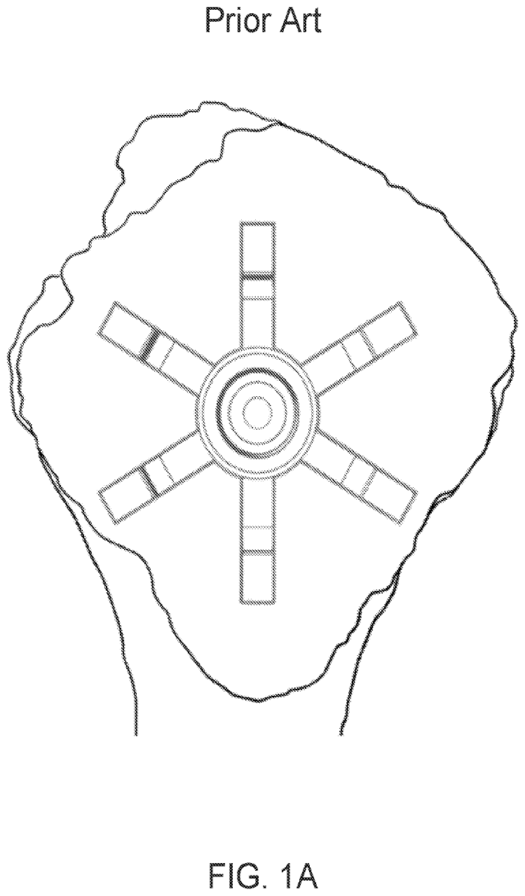

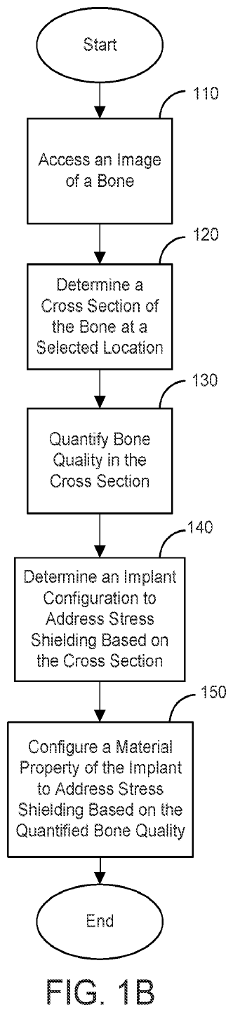

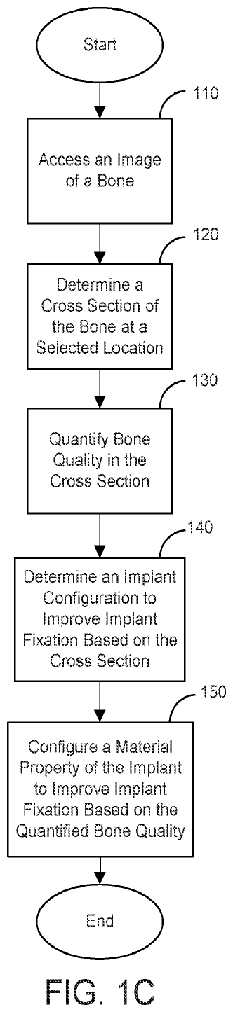

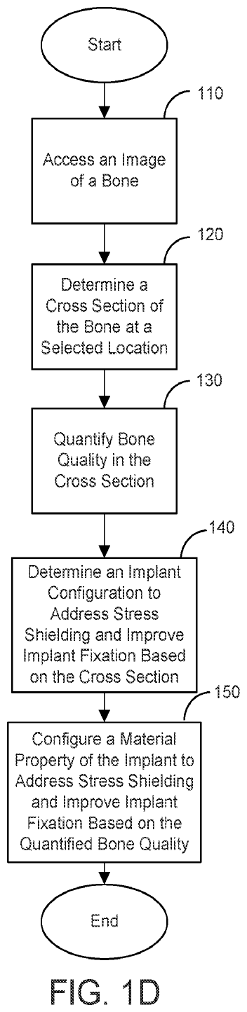

Referring to FIG. 1A, a prior art symmetric implant is shown in cross-section. Previously, the shape, texturing, as well as material properties for implants like the humerus have been symmetric without regard for the relative quality of the cortical or cancellous bone. The central flaw and deficiency of symmetric implants is that the implants do not match the anatomy. Anatomy is not symmetric. There are dramatic differences in the thickness of cortical and cancellous bone based on the specific location within the bone.

In accordance with the present disclosure, an implant shape can be adjusted by applying porous ingrowth coating either thinner or thicker in specific regions to address stress shielding. In another configuration, the shape of the fins of an implant may be adjusted in specific regions of the implant to address stress shielding. In another configuration, the fin length of the implant may be adjusted to engage the best quality peripheral bone. These methods can be used alone or in combination. Additional methods that could be used alone or together with adjusting the coating thickness, implant shape, and fin length include: adjusting implant material elasticity or modulus of elasticity, using different types of coating on the same implant, different porosity of the coating on the same implant, different surface coating treatments, different surface coating shapes; and the like.

The shape of the implant has the potential to increase the immediate fixation and pull out strength of the implant. Additionally, when the fins are made of different lengths to better engage the best quality peripheral bone, this also has the potential to further increase the immediate pull out strength. Moreover, the strategies discussed as a part of the methodology can improve long term fixation by decreasing stress shielding.

In some configurations, cortical and cancellous bone quality can be determined in cross-sections at any location or interval down a bone. Determination of bone quality can also be performed in any angular direction from the center of the bone. With such measurements, a true three dimensional quantification of cortical and cancellous bone quality in a 3-dimensional assessment may be possible for a bone. The shape and material properties of the implant may then be tailored to match the three dimensional architecture of the bone and/or to target the highest identified bone quality in order to address stress shielding.

In a non-limiting example, images of a bone may be divided into specific levels. At each level, specific zones may be defined. In some configurations, the quality of the bone in each zone may be quantified. This quantification may be performed using a medical imaging system, such as radiography, CT scan, densitometry, dual energy x-ray absorptiometry (DEXA), and the like. In each zone, a user or an automated system may score the material property of the bone by using the quality of the specific component of the bone (cortical and/or cancellous). This score may then guide the material properties in a corresponding specific region of the implant. The methodology allows the implant to better match the underlying bone with more uniform stress distribution by customizing the implant or a coating of the implant at each corresponding location of the bone. This information can be used to customize the implant for an individual patient. This information could also be generated on a population of patients and implants can then be tailored to specific patient populations.

Referring to FIG. 1B, non-limiting example steps are shown for a method for adapting an implant to address stress shielding in a bone of a subject by targeting quality bone for implantation. An image of a bone may be obtained from an image archive, acquired using a medical imaging system, or otherwise accessed at step 110. Cross sections of the bone may be determined at step 120 for selected locations along the bone. The quality of a bone (e.g. quantity, strength, quality, and the like), such as cortical bone or cancellous bone, may be determined for the cross sections at step 130, or for select locations within the cross sections. An implant configuration to address stress shielding, such as shape, geometry, number of fins, fin length, orientation of fins or arms, and the like, may be determined at step 140 for the cross sections. In a non-limiting example, the determined bone quality may be combined with measurements from a defined location of the bone to determine an implant configuration, such as shape, geometry, number of fins, fin length, orientation of fins or arms, and the like. A material property of the implant to address stress shielding may also be configured at step 150 based on the quantified bone quality. A material property of an implant may include elasticity, surface coating treatment, porosity and the like, to address stress shielding in the bone.

Referring to FIG. 1C, another non-limiting example steps are shown for a method for adapting an implant to improve implant fixation in a bone of a subject by targeting quality bone for implantation. An image of a bone may be obtained from an image archive, acquired using a medical imaging system, or otherwise accessed at step 110. Cross sections of the bone may be determined at step 120 for selected locations along the bone. The quality of a bone (e.g. quantity, strength, quality, and the like), such as cortical bone or cancellous bone, may be determined for the cross sections at step 130, or for select locations within the cross sections. An implant configuration to improve implant fixation such as shape, geometry, number of fins, fin length, orientation of fins or arms, and the like, may be determined at step 140 for the cross sections. In a non-limiting example, the determined bone quality may be combined with measurements from a defined location of the bone to determine an implant configuration, such as shape, geometry, number of fins, fin length, orientation of fins or arms, and the like. A material property of the implant to improve implant fixation may also be configured at step 150 based on the quantified bone quality. A material property of an implant may include elasticity, surface coating treatment, porosity and the like, to address stress shielding in the bone.

Referring to FIG. 1D, non-limiting example steps are shown for a method for adapting an implant to address stress shielding and improve implant fixation in a bone of a subject by targeting quality bone for implantation. An image of a bone may be obtained from an image archive, acquired using a medical imaging system, or otherwise accessed at step 110. Cross sections of the bone may be determined at step 120 for selected locations along the bone. The quality of a bone (e.g. quantity, strength, quality, and the like), such as cortical bone or cancellous bone, may be determined for the cross sections at step 130, or for select locations within the cross sections. An implant configuration to address stress shielding and improve implant fixation, such as shape, geometry, number of fins, fin length, orientation of fins or arms, and the like, may be determined at step 140 for the cross sections. In a non-limiting example, the determined bone quality may be combined with measurements from a defined location of the bone to determine an implant configuration, such as shape, geometry, number of fins, fin length, orientation of fins or arms, and the like. A material property of the implant to address stress shielding and improve implant fixation may also be configured at step 150 based on the quantified bone quality. A material property of an implant may include elasticity, surface coating treatment, porosity and the like, to address stress shielding in the bone.

In a non-limiting example, quantifying bone quality includes determining a bone quality score, with higher scores indicating higher bone quality. Lower bone quality may be reflected by a lower score. In a non-limiting example, a scoring range can then be obtained with 1 being low and 10 being high.

In one configuration, a score that quantifies the quality of the bone may be determined and include a determination of the location in a cross section of the bone. Different radial locations in the same cross section of the bone may have different scores and therefore would have differing amounts of intervention to address stress shielding, such as bone in-growth coatings to an implant and the like.

In another configuration, a score may be determined qualitatively using a clinical feedback that assesses the quality of the bone based upon a location in a cross section of the bone and clinical knowledge of the stress shielding effects to be expected. Different radial locations in the same cross section of the bone may have different scores and therefore would have differing amounts of intervention to address stress shielding, such as through application of bone in-growth coatings to an implant, location of fins on the implant, and the like.

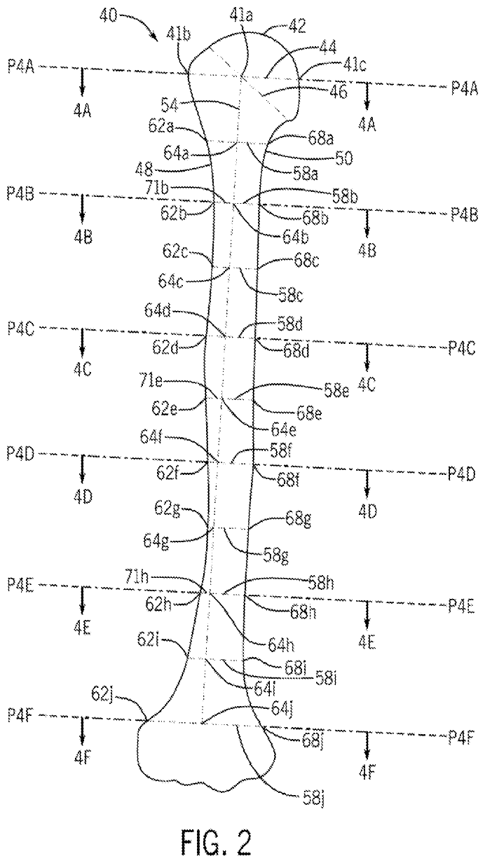

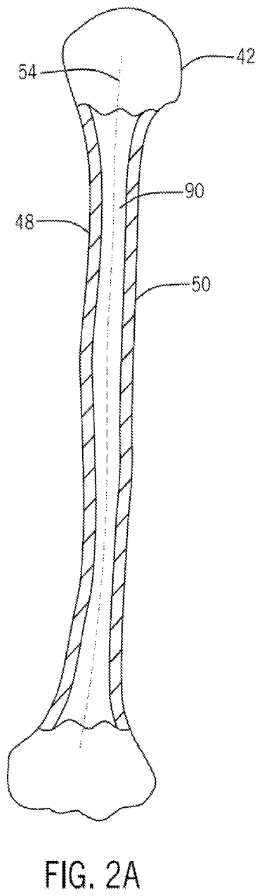

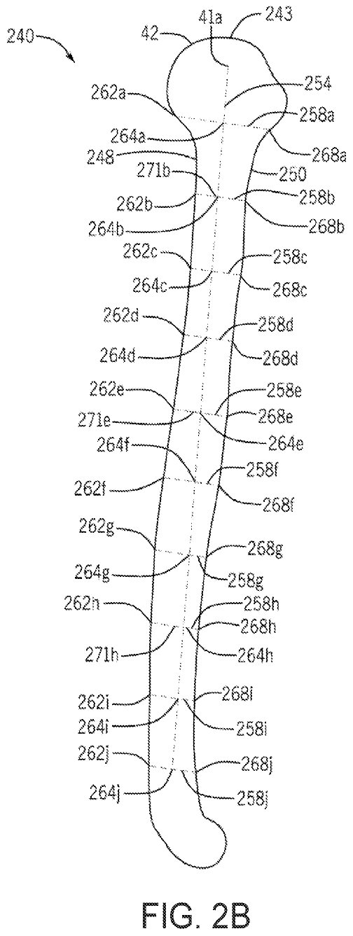

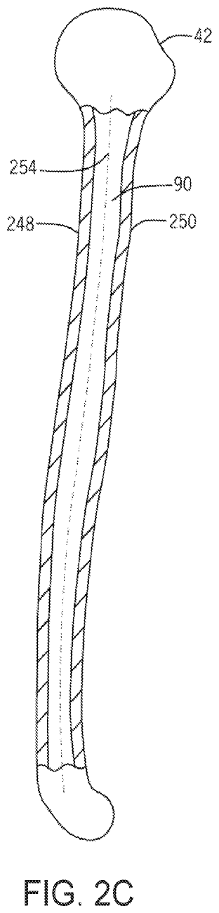

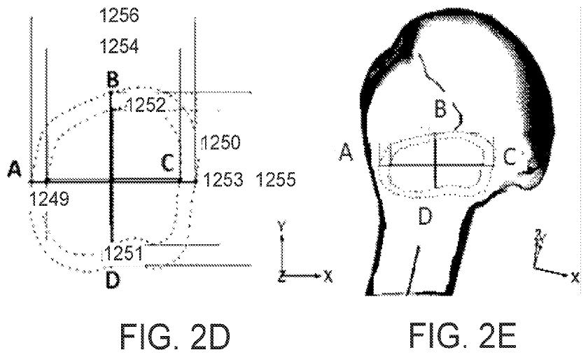

Looking at FIGS. 2-2E, the anatomic shape of a bone and thereby the asymmetric configuration for an implant can be determined by a number of steps. Referring to FIG. 2, an image 40 of a bone 42 of a subject can be obtained, where in some embodiments the image 40 can be a CT image, in other embodiments the image can be an X-ray image, an ultrasonic image, a magnetic resonance image (MRI), a positron emission tomography (PET) image, or the like. The bone can be a femur, tibia, fibula, and the like. In other embodiments, the bone can be a radius, an ulna, or any other bone. A bone cut line 46 can be oriented on the image 40 that can extend from a first border 48 of the bone 42 to an opposite second border 50 of the bone 42. In some embodiments, the bone cut line 46 can be oriented angularly across a region of a head of the bone of a subject. A longitudinal bone axis 54 can be oriented on the image 40. The longitudinal bone axis 54 may extend longitudinally from a proximal aspect of the bone. In another embodiment, the longitudinal bone axis 54 may extend longitudinally from an intersection 41A of a proximal aspect line, such as proximal bone head line 44 with the bone cut line 46, where the proximal bone head line 44 is oriented on the image 40 by extending perpendicularly from a first intersection point 41B on the first border 48 of the bone 42 at the most proximal and lateral aspect of a greater tuberosity through a second intersection point 41A where the proximal bone head line 44 intersects the bone cut line 46, and further extends to a third intersection point 41C on the second border 50 of the bone 42. In some embodiments, the bone 42 can be the femur, or the tibia, or the fibula.

Referring to FIG. 2A, one embodiment is shown where the longitudinal bone axis 54 can follow the centerline of bone 42, defined as being a constant equal distance between the first border 48 and the second border 50. When bone axis 54 is the centerline of the bone 42, the nonlinear shape of the axis line 54 defines the radius of curvature for the bone 42, which can be assessed at various points along the length of the bone axis line 54. The nonlinear shape of the axis line 54 can provide a number of different radii of curvature. When bone axis 54 is the centerline, the intersection of axis line 54 with proximal bone head line 44 may determine intersection point 41A. Any number of changes in radius of curvature can be provided such that the axis line 54 is a constant equal distance between the first border 48 and the second border 50 within the intramedullary canal 90 of the cancellous bone.

Referring again to FIG. 2, in another embodiment the longitudinal bone axis 54 does not follow the centerline, but may be linear and can extend from the bone cut line 46, or may extend linearly from intersection point 41A that was established from the intersection of a centerline with proximal greater tuberosity line 44, along a length of the bone between the first border 48 and second border 50. A plurality of lateral lines 58a, 58b, 58c, 58d, 58e, 58f, 58g, 58h, 58i, 58j can be oriented on the image 40 at different distances from the intersection point 41a, or from a proximal aspect line, such as proximal greater tuberosity line 44, or from the bone cut line 46. Each of the plurality of lateral lines 58a to 58j can extend perpendicularly from one of a plurality of first intersection points 62a, 62b, 62c, 62d, 62e, 62f, 62g, 62h, 62i, 62j on the first border 48 of the bone 42 to one of a plurality of second intersection points 64a, 64b, 64c, 64d, 64e, 64f, 64g, 64h, 64i, 64j intersecting the longitudinal bone axis 54 at one of a plurality of second intersection points 64a to 64j. Each of the plurality of lateral lines 58a to 58j can further extend perpendicularly from one of a plurality of second intersection points 64a to 64j on the longitudinal bone axis 54 to one of a plurality of third intersection points 68a, 68b, 68c, 68d, 68e, 68f, 68g, 68h, 68i, 68j on the second border 50 of the bone 42. The anatomic shape of the bone 42 can be extrapolated based on determining the first intersection point 41B of the proximal greater tuberosity line 44 along with the plurality of first intersection points 62a to 62j, and measuring the distances to the corresponding second intersection points, which for intersection point 41B would be intersection point 41A of the proximal greater tuberosity line 44, and subsequently the plurality of second intersection points 64a to 64j from the first intersection points 62a to 62j. Specifically, the anatomic shape of the first border 48 of the bone 42 can be extrapolated from the first intersection point 41B of the proximal greater tuberosity line 44 and with the plurality of first intersection points 62a to 62j with the plurality of second intersection points 41a and 64a to 64j. The anatomic shape of the second border 50 of the bone 42 can be extrapolated in a similar manner as above by using the third intersection point 41C of the proximal greater tuberosity line 44 with the plurality of third intersection points 68a to 68j and measuring the distances to the corresponding second intersection points 41a and 64a to 64j.

In some embodiments, the plurality of lateral lines 58a to 58j can be placed at equidistant intervals distally from intersection point 41a, or from a proximal aspect line, such as proximal greater tuberosity line 44, or from the bone cut line 46. In some embodiments, the equidistant interval can be in a range from 0.1 to 50 millimeters. In a non-limiting embodiment, the equidistant interval can be 25 millimeters. As such, example measurements can be made at 25 millimeters, 50 millimeters, 75 millimeters, 100 millimeters, 125 millimeters, 150 millimeters, 175 millimeters, and 200 or more millimeters distal to the intersection point 41a, or from a proximal aspect line, such as proximal greater tuberosity line 44, or from the bone cut line 46. One can add more lines to provide for determining the contour of the bone with higher resolution.

In a non-limiting example embodiment, a first reference distance can be measured for a first line 71b extending perpendicularly from a first point 62b of the plurality of first intersection points 62a to 62h to a first point 64b of the plurality of second intersection points 64a to 64h. A second reference distance can be measured of a second line 71e extending perpendicularly from a second point 62e of the plurality of first intersection points 62a to 62h to a second point 64e of the plurality of second intersection points 64a to 64h. A third reference distance can be measured of a third line 71h extending perpendicularly from a third point 62h of the plurality of first intersection points 62a to 62h to a third point 64h of the plurality of second intersection points 64a to 64h.

The anatomic shape of the first border 48 can be extrapolated based on the first reference distance of the first line 71b, the second reference distance of the second line 71e, and the third reference distance of the third line 71h. A first curvature of the anatomic shape can be extrapolated between the first point 62b of the plurality of first intersection points 62a to 62h and the second point 62e of the plurality of first intersection points 62a to 62h based on the first reference distance and the second reference distance. A second curvature of the anatomic shape can be extrapolated between the second point 62e of the plurality of first intersection points 62a to 62h and the third point 62h of the plurality of first intersection points 62a to 62h based on the second reference distance and the third reference distance.

In another version of the method of the disclosure, the anatomic shape of the first border 48 and the second border 50 together can be extrapolated based on a fourth reference distance of the lateral line 58b, a fifth reference distance of the lateral line 58e, and a sixth reference distance of the lateral line 58h. A first curvature of the anatomic shape can be extrapolated between the first point 62b of the plurality of first intersection points 62a to 62j and the second point 62e of the plurality of first intersection points 62a to 62j based on the fourth reference distance and the fifth reference distance. A second curvature of the anatomic shape can be extrapolated between the second point 62e of the plurality of first intersection points 62a to 62j and the third point 62h of the plurality of first intersection points 62a to 62j based on the fifth reference distance and the sixth reference distance.

Looking at FIG. 2B, the anatomic shape can be determined by a number of steps. An image 240 of a bone 42 of a subject can be obtained in a sagittal viewing plane; in some embodiments the image 240 can be a CT image; in other embodiments the image can be an X-ray image, an ultrasonic image, a magnetic resonance image (MRI), a positron emission tomography (PET) image, or the like. The bone 42 can be a humerus. In other embodiments the bone can be a radius, an ulna, a femur, a tibia, or any other bone.

A longitudinal bone axis 254 can be oriented on the image 240. The longitudinal bone axis 254 may extend from intersection point 41a from FIG. 2 along a length of the bone 42 between a first border 248 and a second border 250. FIG. 2C shows one embodiment where the longitudinal bone axis 254 can follow the centerline of bone 42, defined as being a constant equal distance between the cortical bone first border 248 and the second border 250. Alternatively, longitudinal bone axis 254 may be the centerline of bone 42, being defined as a constant equal distance between the borders of the cancellous bone, which would take into account any differences with cortical bone thickness. When bone axis 254 is the centerline of the bone 42, the nonlinear shape of the axis line 254 defines the radius of curvature for the bone 42, which can be assessed at various points along the length of the bone axis line 254. The nonlinear shape of the axis line 254 can provide a number of different radii of curvature. In one non-limiting example, a first radius of curvature can transition to a second radius of curvature, and the second radius of curvature can transition to a third radius of curvature. The first radius of curvature and the third radius of curvature can be concave, while the second radius of curvature can be convex. Each radius of curvature can feature a different radius. Any number of changes in radius of curvature can be provided such that the axis line 254 is a constant equal distance between the first border 248 and the second border 250 within the intramedullary canal 90 of the cancellous bone. In one embodiment, the deviation from the straight longitudinal bone axis 254 (FIG. 2B) from the centerline following bone axis 254 (FIG. 2C) may be determined in order to indicate where over the length of the bone the area or areas of greatest bending or deflection take place. This may be used when designing plates, intramedullary nails, stems, or other implants for bends that may be needed to conform to the anatomy, and in guiding the locations for where stress shielding may be more likely to occur.

A plurality of lateral lines 258a, 258b, 258c, 258d, 258e, 258f, 258g, 258h, 258i, 258j can be oriented on the image 240 at different distances from the proximal end 243, or from point 41a. Each of the plurality of lateral lines 258a to 258j can extend perpendicularly from one of a plurality of first intersection points 262a, 262b, 262c, 262d, 262e, 262f, 262g, 262h, 262i, 262j on the first border 248 of the bone 42 to one of a plurality of second intersection points 264a, 264b, 264c, 264d, 264e, 264f, 264g, 264h, 264i, 264j intersecting the longitudinal bone axis 254 at one of a plurality of second intersection points 264a to 264j. Each of the plurality of lateral lines 258a to 258j can further extend perpendicularly from one of a plurality of second intersection points 264a to 264j on the longitudinal bone axis 254 to one of a plurality of third intersection points 268a, 268b, 268c, 268d, 268e, 268f, 268g, 268h, 268i, 268j on the second border 250 of the bone 42. The anatomic shape of the bone 42 can be extrapolated based on the plurality of first intersection points 262a to 262j, and the plurality of second intersection points 264a to 264j. Specifically, the anatomic shape of the first border 248 of the bone 42 can be extrapolated from the plurality of first intersection points 262a to 262j and the plurality of second intersection points 264a to 264j. The anatomic shape of the second border 250 of the bone 42 can be extrapolated from the plurality of second intersection points 264a to 264j and the plurality of third intersection points 268a to 268j.

In some embodiments, the plurality of lateral lines 258a to 258j can be placed at equidistant intervals distally from the proximal end 243. In some embodiments, the equidistant interval can be in a range from 0.1 to 50 millimeters. In a non-limiting embodiment, the equidistant interval can be 25 millimeters. As such, example measurements can be made at 25 millimeters, 50 millimeters, 75 millimeters, 100 millimeters, 125 millimeters, 150 millimeters, 175 millimeters, and 200 or more millimeters distal to the proximal end 243. One can add more lines to provide for determining the contour of the bone with higher resolution.

In a non-limiting example embodiment, a first reference distance can be measured for a first line 271b extending perpendicularly from a first point 262b of the plurality of first intersection points 262a to 262h to a first point 264b of the plurality of second intersection points 264a to 264h. A second reference distance can be measured of a second line 271e extending perpendicularly from a second point 262e of the plurality of first intersection points 262a to 262h to a second point 264e of the plurality of second intersection points 264a to 264h. A third reference distance can be measured of a third line 271h extending perpendicularly from a third point 262h of the plurality of first intersection points 262a to 262h to a third point 264h of the plurality of second intersection points 264a to 264h.

The anatomic shape of the first border 248 can be extrapolated based on the first reference distance of the first line 271b, the second reference distance of the second line 271e, and the third reference distance of the third line 271h. A first curvature of the anatomic shape can be extrapolated between the first point 262b of the plurality of first intersection points 262a to 262h and the second point 262e of the plurality of first intersection points 262a to 262h based on the first reference distance and the second reference distance. A second curvature of the anatomic shape can be extrapolated between the second point 262e of the plurality of first intersection points 262a to 262h and the third point 262h of the plurality of first intersection points 262a to 262h based on the second reference distance and the third reference distance.

In some configurations, the anatomic shape of the first border 248 and the second border 250 together can be extrapolated based on a fourth reference distance of the lateral line 258b, a fifth reference distance of the lateral line 258e, and a sixth reference distance of the lateral line 258h. A first curvature of the anatomic shape can be extrapolated between the first point 262b of the plurality of first intersection points 262a to 262j and the second point 262e of the plurality of first intersection points 262a to 262j based on the fourth reference distance and the fifth reference distance. A second curvature of the anatomic shape can be extrapolated between the second point 262e of the plurality of first intersection points 262a to 262j and the third point 262h of the plurality of first intersection points 262a to 262j based on the fifth reference distance and the sixth reference distance.

Referring to FIG. 2D, the plurality of lateral lines 58a, 58b, 58c, 58d, 58e, 58f, 58g, 58h, 58i, 58j from FIG. 2 or FIG. 2A may include being placed in multiple planes, such as A-C and D-B in FIG. 2D. The plurality of lateral lines may also include measurements of the thickness of the cortical and cancellous bone material. In the example provided, thicknesses for the cortical and cancellous bone is obtained in a 2D cross section of the humerus, such as the cross section depicted in FIG. 2E. Cortical lateral thickness 1249, cortical medial thickness 1250, cortical anterior thickness 1251, and cortical posterior thickness 1252 may be determined. Cancellous anterior to posterior distance 1253, cancellous medial to lateral distance 1254, cortical anterior to posterior distance 1255, and cortical medial to lateral distance 1256 may also be determined. In some embodiments, any orientation for the planes may be used, such as a partially rotated anterior to a partially rotated posterior view, which may enable for fully 3D thickness measurements of the bone. In one embodiment, these measurements may be obtained in an automated fashion, where a medical image is provided to a computer system that automatically segments the bone, identifies the relevant anatomical landmarks, such as the humeral head and the olecranon fossa, and performs the desired measurements.

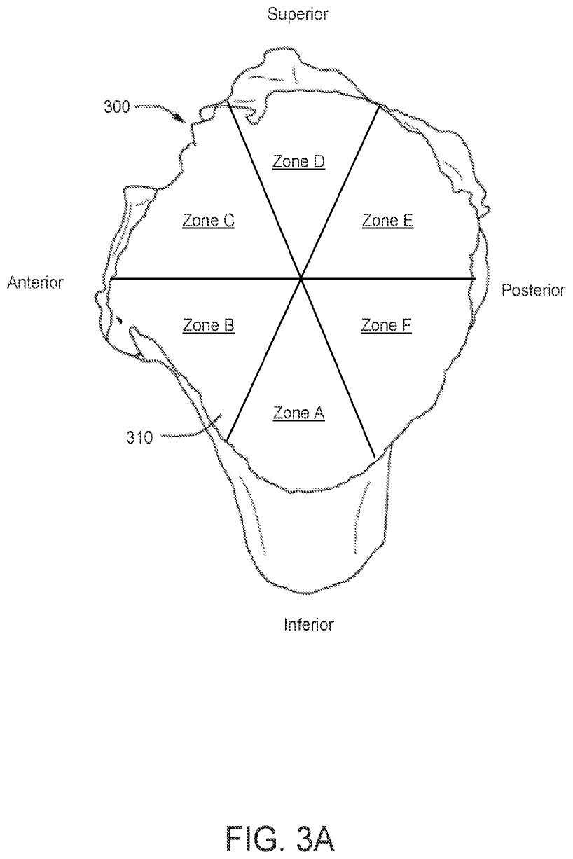

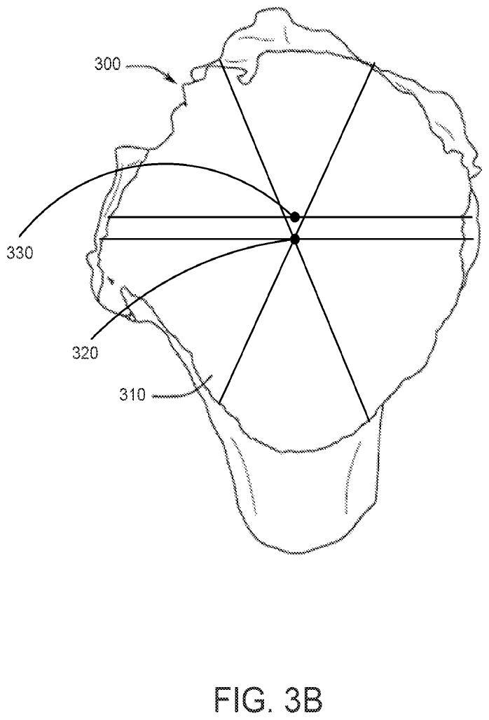

Referring to FIG. 3A, a non-limiting example for bone quality assessment is shown in a cross section of a proximal humerus 300. Scoring of bone quality can be performed for zones in a cross section of a bone and the fins or arms of an implant may be optimized for the bone quality in each zone. A bone quality zone 310 may be applied as radial sections of the cross section, as shown. Zones A-F are shown in the non-limiting example as bone quality zones. In a non-limiting example, a zone, such as zone A, may be identified as having higher bone quality and an implant arm length, arm thickness, and/or coating thickness may be increased in a portion of the implant corresponding to zone A in order to address stress shielding or improve implant fixation. One skilled in the art would appreciate that any number of zones may be used, and any geometric configuration of zones (e.g. rectangular sections, circular sections, and the like) may be used. A score may be applied to each zone used, such as shown in Table 1.

TABLE 1
Proximal Humerus Bone Quality

| Zone | Central Bone | Peripheral Bone |
|---|---|---|
| A | 2 | 10 |
| B | 2 | 10 |
| C | 2 | 8 |
| D | 2 | 5 |
| E | 2 | 6 |
| F | 2 | 8 |

Referring to FIG. 3B, the non-limiting example for bone quality assessment of a proximal humerus 300 of FIG. 3A is shown at the level of a humeral cut. The humeral cut shows the potential difference in locations for a reverse cut vertical midpoint 320 and anatomical cut vertical midpoint 330. There is also a distribution of bone quality in the proximal humerus at the level of a humeral cut for shoulder arthroplasty. The highest bone density in the proximal humerus is typically in the inferior and anterior region. The poorest quality humeral bone is in the superior region. The bone density in the proximal humerus at the level of a humeral head cut is also significantly higher peripherally compared to centrally. In addition, the denser peripheral bone has been shown to have greater biologic activity and osteointegration potential compared to central bone. Methods in accordance with the present disclosure may be used to differentially engage the better quality inferior and anterior bone as well as the peripheral bone for both immediate as well as long term fixation.

Finite element and clinical research has demonstrated that changes in bone stress in the proximal humerus with stemless implants are most prominent in the superior humeral region. This is consistent with the superior region also having the poorest quality bone. Thinner peripheral fins on stemless implants have been shown to produce less stress changes than thicker peripheral fins. This may be due to these thinner fins being less rigid, resulting in less stress shielding compared with thicker peripheral designs. To minimize stress shielding, an implant may be configured with thinner fins superiorly and then progressively thicker fins in regions with better bone quality. An implant may also be configured to have the fins thinner in the central region where there is poorer quality bone and wider more peripherally to engage the better quality peripheral bone to decrease stress shielding.

The implant shape can be adjusted by applying porous ingrowth coating either thinner or thicker in specific regions. Keeping the base implant the same and adjusting only the coating thickness may have advantages in regard to inventory and accelerated regulatory approval.

In a non-limiting example, thinner fins may be used in regions of poorer bone quality, and thicker fins may be used in regions of better bone quality. The thickness of the fin may be proportional to the bone quality. In another non-limiting example, an inverse configuration may be provided where the thickness of the fin may be inversely proportional to the bone quality, such that thinner fins may be used in regions of better bone quality and thicker fins used in regions of poorer bone quality.

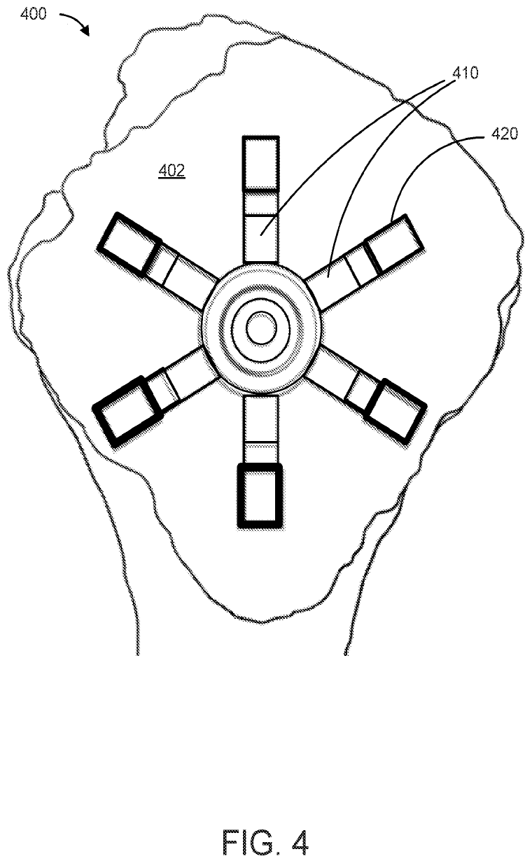

Referring to FIG. 4, a non-limiting example of an asymmetric coating implant 400 is shown in an example proximal humerus cross section 402. The underlying thickness of the fins 410 are the same. The coating thickness 420 is applied asymmetrically to the implant in order to address stress shielding, such that different fin locations have different coating thicknesses 420. The coating thickness 420 is adjusted on the peripheral aspect of the fins. The coating thickness is proportionate to the bone quality with the poorest superior region having the thinnest coating, then posterior-superior being thicker, followed by anterior-superior and posterior-inferior, followed with the best quality inferior and anterior-inferior peripheral fins having the thickest coating. As shown, the outer part of the fins look wider. One method for asymmetric coating is to apply layers of coating to the outer parts of the fins to create this asymmetric implant shape. In some configurations, there is no need to taper the fins, as the coating creates the desired asymmetry. In some configurations, the edges can be sharp.

Referring again to FIG. 3B, after a humeral head cut is made for anatomic shoulder arthroplasty, the proximal humerus is not symmetrically shaped. It is more heart shaped with narrowing inferiorly. Additionally, due to the shape of the lesser tuberosity located anteriorly, the proximal humerus becomes narrower anteriorly compared to posteriorly. When performing a reverse shoulder arthroplasty, a lower humeral cut is typically made. The changes in the shape of the proximal humerus become even more pronounced. This can be seen in the difference between locations for a reverse cut vertical midpoint 320 and anatomical cut vertical midpoint 330 in FIG. 3B. To engage the best quality peripheral bone, an implant may be configured with fins of different lengths to better engage the peripheral bone.

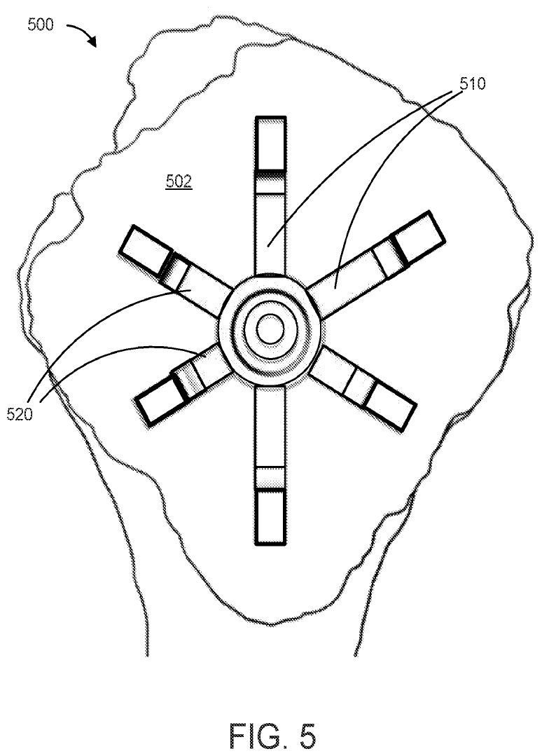

Referring to FIG. 5, a non-limiting example of an asymmetric fin length implant 500 is shown in proximal humerus cross section 502. Asymmetric fin length implant 500 includes long fins 510 and short fins 520 spaced asymmetrically about the implant. Asymmetric fin length implant 500 has a uniform underlying thickness of fins, uniform coating thickness, but includes a variable fin length to engage the best quality peripheral bone.

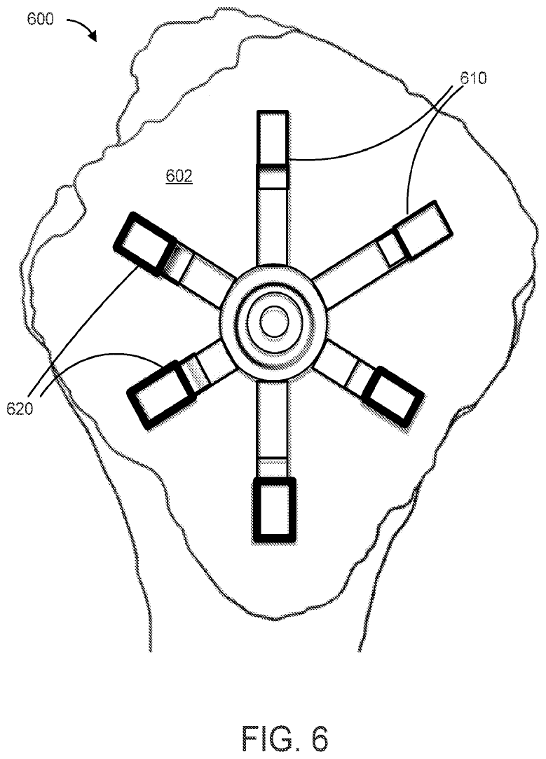

Referring to FIG. 6, a non-limiting example of an asymmetric fin length implant with asymmetric coating 600 is shown in proximal humerus cross section 602. Asymmetric fin length implant with asymmetric coating 600 has a uniform underlying thickness of fins, but includes variable coating thickness noted by lesser coating thickness 610 on certain fin lengths, and greater coating thickness 620 on other fin lengths. In a similar manner to FIG. 5, the length of each of the fins is optimized to engage the best quality peripheral bone. Additionally, the coating thickness 610 and 620 is proportionate to the bone quality with the poorest superior region having the thinnest coating and the best quality inferior and anterior peripheral fins having the thickest coating. As noted previously, the bone quality is significantly poorer centrally compared to peripherally, so the fins may be configured to be thinner centrally compared to peripherally.

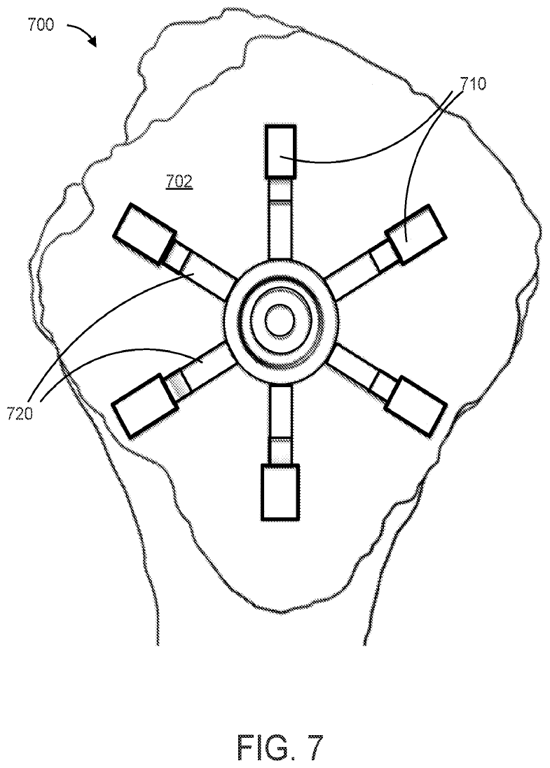

Referring to FIG. 7, a non-limiting example of an asymmetric fin thickness implant 700 is shown in proximal humerus cross section 702. Asymmetric fin thickness implant 700 has variable thickness of the peripheral fins 710 as compared to more central fins 720, but with uniform coating, and uniform fin length. The thickness of the peripheral fins 710 is proportionate to the bone quality with the poorest superior region having the thinnest peripheral fin, then posterior-superior being thicker, followed by anterior-superior and posterior-inferior, followed with the best quality inferior and anterior-inferior peripheral fins having the thickest peripheral fins.

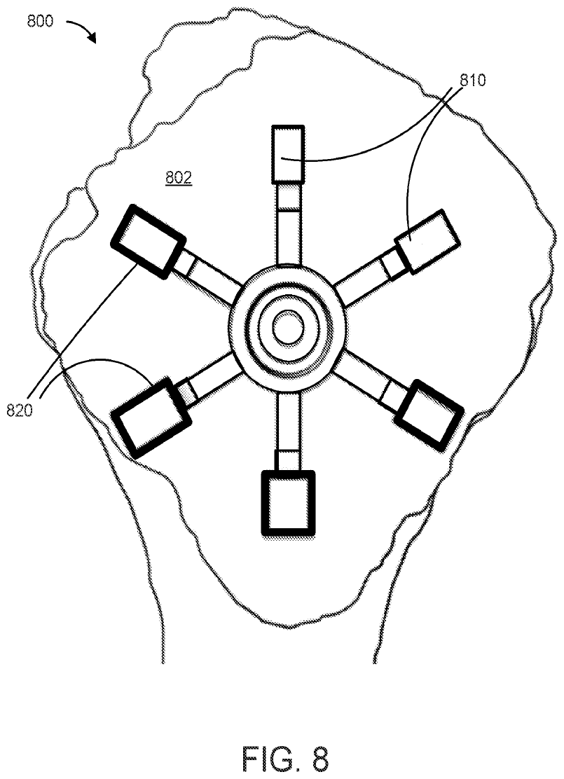

Referring to FIG. 8, a non-limiting example of an asymmetric fin thickness with asymmetric coating thickness implant 800 is shown in proximal humerus cross section 802. Asymmetric fin thickness with asymmetric coating thickness implant 800 has variable thickness of the peripheral fins 810 with variable coating thickness 820, and uniform fin length. The thickness of the peripheral fins 810 and thickness of the coating 820 may be proportionate to the bone quality in those locations.

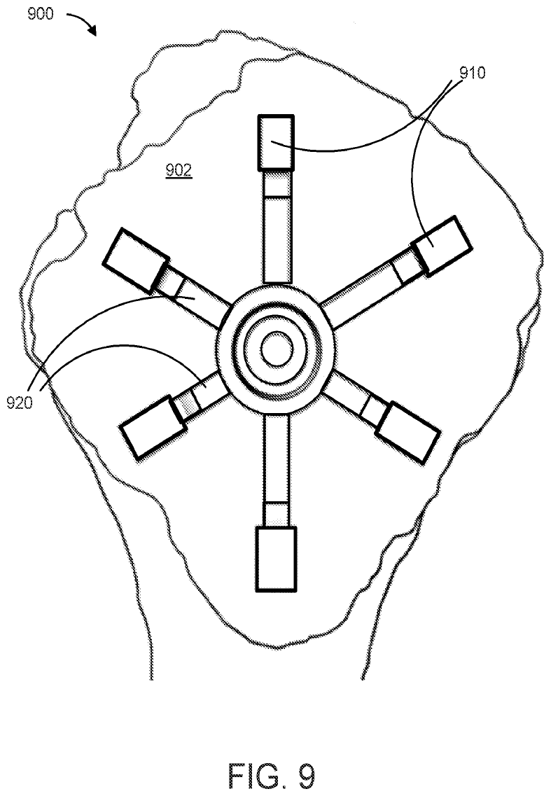

Referring to FIG. 9 a non-limiting example of an asymmetric fin thickness with asymmetric fin length implant 900 is shown in proximal humerus cross section 902. Asymmetric fin thickness with asymmetric fin length implant 900 has variable thickness of the peripheral fins 910, uniform coating thickness, and variable fin length 920. The thickness of the peripheral fins 910 may be proportionate to the bone quality. The length of each of the fins 920 may be optimized to engage the best quality peripheral bone.

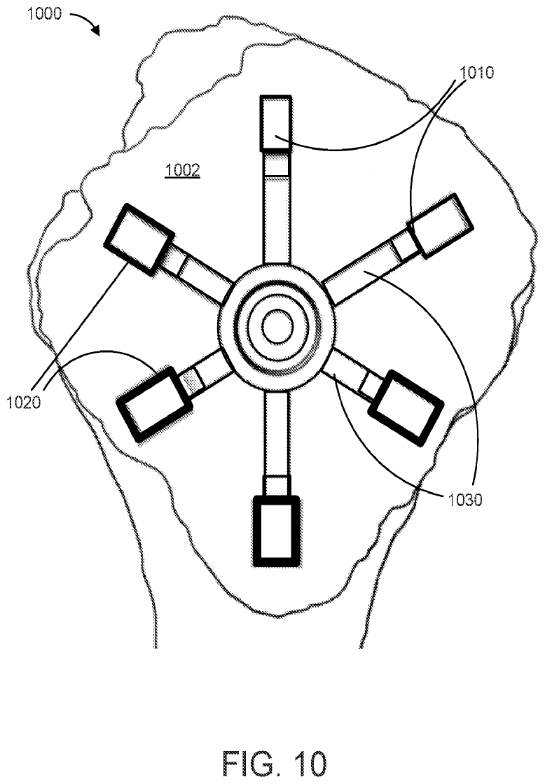

Referring to FIG. 10 a non-limiting example of an asymmetric fin thickness with asymmetric fin length and asymmetric coating thickness implant 1000 is shown in proximal humerus cross section 1002. Asymmetric fin thickness with asymmetric fin length and asymmetric coating thickness implant 1000 has a variable thickness of the peripheral fins 1010, variable coating thickness 1020, and variable fin length 1030. The thickness of the peripheral fins 1010 and coating thickness 1020 may be proportionate to the bone quality. The length of each of the fins 1030 may be optimized to engage the best quality peripheral bone.

Non-limiting example stemless proximal humeral implants have been depicted, but implants may take on any form including, but not limited to, joint arthroplasty, trauma, dental implants, and the like. Non-limiting examples include stems for joint arthroplasty, stemless implants for joint arthroplasty, trauma implants including intramedullary nails and plates, sports medicine implants such as anchors and screws, as well as dental implants.

The material properties of the implant, such as a plate, intramedullary nail, and the like, can be tailored to the specific anatomic site. The thickness of the cortices varies significantly based on the specific anatomic location where a plate may be placed. An implant designed with these factors taken into account can result in more physiologic loads to the underlying bone and has the potential to minimize stress shielding and improve fracture healing. Moreover, the material properties of an implant, such as an intramedullary nail may be optimized for patients with different quality bone in specific locations, such as the difference between young patients compared to older patients. This has the potential for wide implementation since the distribution of many fractures constitutes a clear bimodal population. Femur fractures occur most frequently in either high velocity accidents in young males or simple falls in elderly women.

Implant elasticity and the long-term bone integrity associated with adaptive bone remodeling are strongly related. Altering implant material properties can alter elastic properties. Adjusting titanium porosity results in elastic properties closer to those of bone than to solid titanium surfaces. Functionally graded material (FGM), where the mechanical properties can be tailored to vary with position within the material, may be used to adjust the modulus of elasticity within an implant.

In some configurations, the modulus of elasticity of the implant may be modified by adjusting the coating of the implant in regards to type, porosity, thickness, and the like with respect to location on the implant. Modification of elasticity may also include using different coatings in different areas of the implant. Modification of elasticity may also include altering the shape of the implant, such as by matching a fluted, oval, spherical, or other shape to the anatomy of the bone. Modification of elasticity may also include adjusting the modulus of elasticity of the implant material in a zone specific manner.

Surface roughness treatments such as polishing, sandblasting, plasma-spraying, or porous beading may have an impact on the distribution of stresses at the bone-implant interface. In some configurations, the use of surface roughness methods may result in reduction of stress shielding in peri-implant tissues. In FEA analysis, porous-surfaced implants were shown to distribute stress in a more uniform pattern around the implant compared with smooth surfaced implants. However, these coatings have traditionally been applied in a symmetric manner. Moreover, in these FEA analysis, bone has been simplified and modeled to be symmetric. In accordance with the present disclosure, surface roughness methods may be applied or utilized asymmetrically on an implant. Imaging or modelling the asymmetry in bone architecture may be used to apply asymmetric coatings to an implant to better match the implant to the anatomy and decrease stress shielding.

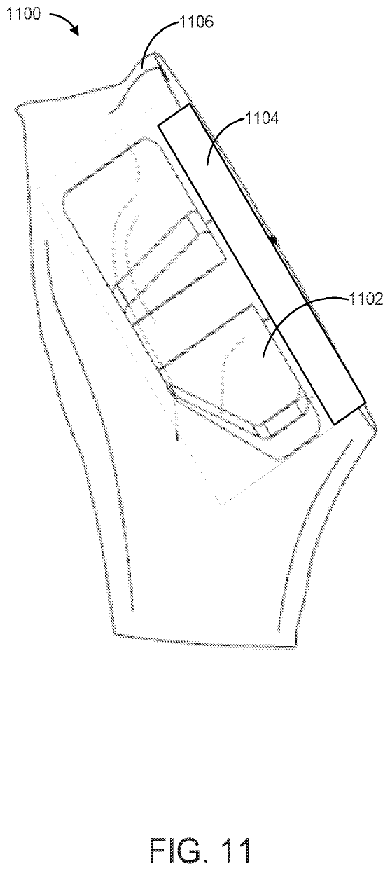

Referring to FIG. 11, a non-limiting example of an inlay implant 1100 implanted in a humerus 1106 is shown. Inlay implant 1100 includes body 1102 that includes fins or other protrusions that may be configured in accordance with the present disclosure. Inlay implant 1100 also includes articulating surface body 1104. For the inlay implant 1100, the body 1102 is positioned within the humerus 1106 such that the articulating surface body 1104 is flush with the surface of the bone.

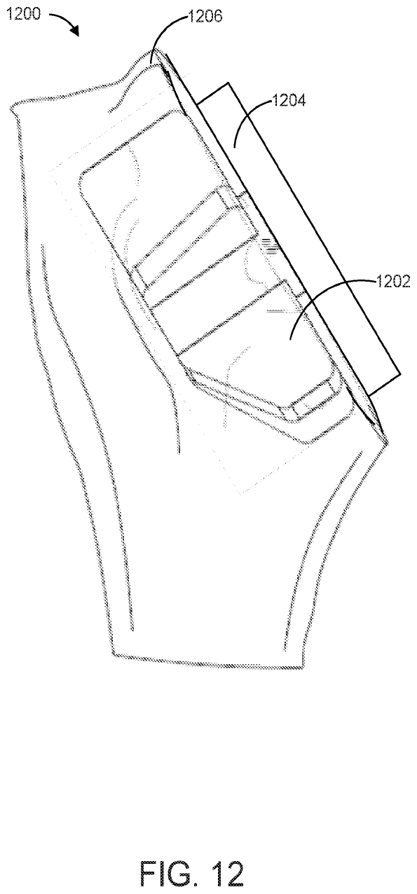

Referring to FIG. 12, a non-limiting example of an onlay implant 1200 implanted in a humerus 1206 is shown. Onlay implant 1200 includes body 1202 that includes fins or other protrusions that may be configured in accordance with the present disclosure. Onlay implant 1200 also includes articulating surface body 1204. For the onlay implant 1200, the body 1202 is positioned within the humerus 1206 such that the articulating surface body 1204 is positioned above the surface of the bone.

### Finite Element Analysis Simulation

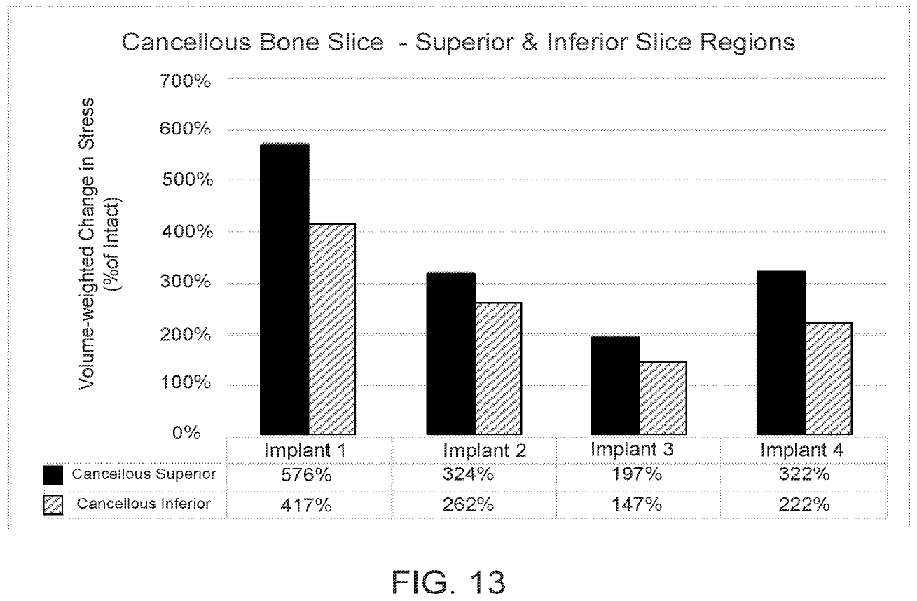

Referring to FIG. 13, non-limiting example results from a simulation protocol are shown in the form of a bar graph of the volume-weighted change in stress by implant model. To assess the potential for stress shielding to occur in stemless humeral implant prototypes, non-limiting example biomechanical finite element simulations were performed. The models were evaluated by using anticipated worst-case loading of 740N at 75° abduction applied to a patient-specific anatomical model. Finite element meshes of both the implant models and anatomical model were created in Materialise 3-matic® software and subsequently imported into Dassault Systems Abaqus® finite element engineering software.

To account for the heterogenous nature of the spatially distributed bone mineral density (BMD), an approach was adopted with BMD in the superior region of the cancellous region set at 0.2 g/cc and the inferior region set at 0.35 g/cc. The mapping between BMD and the elastic modulus used in the simulations was made using experimental testing.

### Implant Ranking Method:

Previous efforts we identified for evaluating stress shielding of implants during design phases undertook computerized methods to analytically predict the likelihood of this behavior. They hypothesized that bone stress in a reconstructed state that was closer to the stress in the intact bone would be less likely to remodel or resorb (from Razfar et al. p. 1080: “For the purpose of this investigation, it is proposed that any changes in bone stress from the intact state are a consequence of joint reconstruction and accordingly should be minimized, as stress changes could lead to a cascade effect whereby stress shielding is initiated”). They go on to state that by “cascade”, some bone would remodel (“strengthen”) due to the presences of higher stress after reconstruction and some would resorb (“weaken”) due to absence of prior stress. Bone that remodels would further propagate the problem over time in the resorbed bone on top of the initial shielding provided by implant. (Basically a “double bogey” for the newly shielded tissue.)

Taking these concepts into account, the modeling results were evaluated by comparing ratio of the average stress (weighted by element volume) in a volume slice of cancellous bone of the implant model to that same slice of bone in the intact model. This method is practical in an engineering sense in that it normalizes the average cancellous bone stress fairly against that of the intact bone. This allows the practitioner to discern the degree of stress change the bone will likely experience when reconstructed with a particular implant, providing a “single metric” means of comparison of implants.

### Results:

The results from the stemless shoulder designs are presented in the bar chart of FIG. 13. Implant 1, which was a conventional, all-uniform implant with the shortest fin lengths, had average stress in the reconstructed state that was 576% of intact in the superior region and 417% of intact in the inferior region. Implant 2 included uniform fin thickness with a variable length for the fins. Implant 3 included peripheral fin thickness proportionate to bone quality with variable length fins. Implant 4 included the inverted configuration where fin thickness was reduced in the higher quality bone portions and also included variable length fins. Implants 2-4 which had fin lengths more peripheral to the axis of the implant had substantially lower stresses, with Implant 3 superior and inferior regions performing most similarly to the intact case (147% and 197%, respectively).

### Biomechanical Testing

A testing protocol was created to quantify resistance to torque-out failure. Each model was implanted in a rigid polyurethane foam with density of 5 PCF (Pacific Research Laboratories, Inc., Vashon Island, Washington), per the guidelines specified by ASTM F-1839-08 “Standard Specification for Rigid Polyurethane Foam for Use as a Standard Material for Testing Orthopaedic Devices and Instruments”.

A cannulated technique was utilized. A guidewire was placed in the foam bone followed by drilling of a center hole. A preparation tool 1 millimeter in width smaller than the model was used to create channels. This allowed for a 1 millimeter interference fit when the model was placed in the foam. Each model was embedded to a depth such that the medial surface of the model was flush with the surface of the foam.

Each embedded model was rigidly mounted to a 6-axis load cell (ATI Mini 58) via a bolt screwing into the central channel of the model such that the inferiorly located fin of the model pointed downward parallel to gravity. A torque replicating that of a shoulder-elevating motion was applied about the anterior-posterior axis until failure. Failure was defined as the model no longer being fixed within the block due to the foam fracturing around the model or the model sliding free of the foam. During this torque-out process, 3-axis force and 3-axis torque values were recorded at 100 Hz. 6 trials were conducted for each model implant and a summary of the results are show in Table 2 below.

TABLE 2
Biomechanical Testing Summary

| | Symmetric | Asymmetric | Asymmetric fin |
|---|---|---|---|
| | fin length and | fin length and | length and |
| | symmetric fin | symmetric fin | asymmetric fin |
| Trial | thickness | thickness | thickness |
| Number | Implant 1 | Implant 2 | Implant 3 |
| 1 | 2.767 | 3.822 | 3.854 |
| 2 | 2.478 | 3.663 | 4.036 |
| 3 | 2.336 | 4.065 | 4.49 |
| 4 | 2.331 | 3.711 | 3.931 |
| 5 | 2.615 | 3.83 | 4.036 |
| 6 | 2.377 | 3.674 | 4.249 |
| Average | 2.48 | 3.79 | 4.10 |

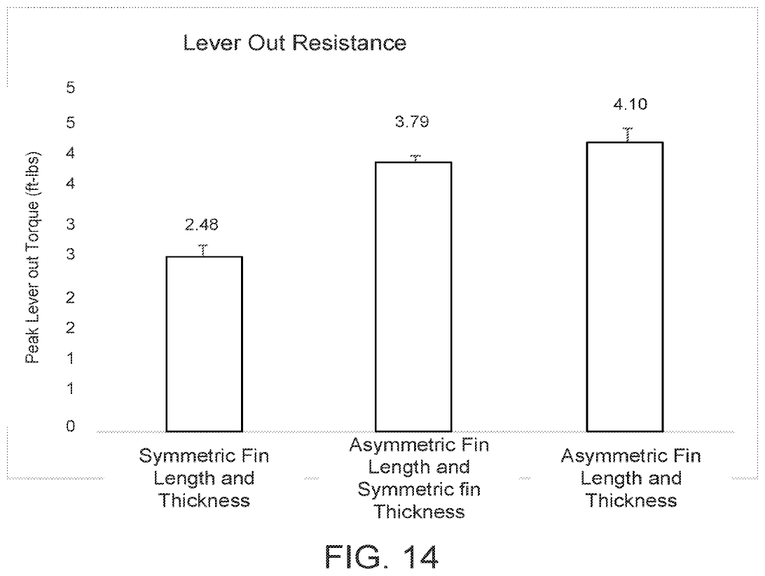

Referring to FIG. 14, the resultant torque at failure for each of three models, averaged across the trials, is shown. These combined torques represent the shoulder loads experienced during humeral elevation. Failure torque was defined as the peak torque during the test. The model implants with asymmetric fin length had significantly greater resistance to torque-out failure than the conventional circular model with symmetric fin length ((Implant 1 vs. Implant 2 (p<0.0001) (Implant 1 vs. Implant 3 (p<0.0001)). Additionally, Implant 3 with asymmetric fin length and asymmetric fin thickness had significantly greater resistance to torque-out failure than Implant 2 with asymmetric fin length and symmetric fin thickness (p<0.05). The testing validated the methodology and demonstrated how anatomic features can be used to guide the modification of implants to distribute optimal loading, prevent stress shielding, improve fit at the bone-device interface, and improve implant fixation.

### Non-Limiting Example Population Based Design

In some configurations, the method utilizes the bone properties measurements of a wide population of patients. Implants may then be manufactured in mass to fit an average size or configuration rather than being patient specific. In some configurations, different implant size and configuration options may be created based on differing populations. In non-limiting examples, an implant may include characteristics optimized for a younger patient with thicker cortices and good quality bone, and another implant may be optimized for an older patient with bone that has thinner cortices and poorer quality bone.

The methods in accordance with the present disclosure may be used to result in a population based average of bone quality for an off the shelf solution. A specific number of left and right specific implants with sizes based on a distribution of anatomy can be manufactured. This could include optimized fin length based on the methodology. In some configurations, a semi-custom implant may be provided, where implant options exist for differing patient populations such as a younger patient with good quality bone compared to an older patient with poorer quality bone.

### Custom Implant Application

Individualized implants may be designed using additive manufacturing and 3D printing to customize the implant for an individual patient in accordance with the present disclosure. The material properties of the implant including coatings, shape, and material properties may be adjusted to create in an implant designed to minimize stress shielding for that individual patient.

The development of personalized implants may be based on the individual anatomy of a patient with optimized fin length as well as fin designs based on their specific bone quality. A variety of implant designs may be used to decrease stress shielding and improve immediate fixation thereby facilitating the design of smaller implant. Implant designs can be optimized from joint arthroplasty (hip, knee, shoulder, elbow, ankle, spine, etc.), trauma implants, as well as dental implants.

The present disclosure has described one or more preferred embodiments, and it should be appreciated that many equivalents, alternatives, variations, and modifications, aside from those expressly stated, are possible and within the scope of the invention.

1. A method for manufacturing an orthopedic implant for repairing a part of a bone in a subject, the method comprising:
forming the implant to include at least one material property determined by;
i) obtaining an image of the bone from at least one viewing plane;
ii) orienting on the image a cross section of the bone;
iii) quantifying a quality of the bone based on the cross section;
iv) determining the at least one material property of the implant to reduce stress shielding for the bone using the quantified bone quality.

2. The method of claim 1, wherein the material property includes at least one of elasticity, surface coating treatment, porosity, thickness, fin length, fin thickness, or implant shape.

3. The method of claim 2, wherein the material property creates an asymmetric implant.

4. The method of claim 2, wherein the at least one material property includes the surface coating treatment of the implant, and wherein the surface coating treatment corresponds to an anatomic location determined by a location of the cross section of the bone.

5. The method of claim 4, wherein the surface coating treatment is asymmetric on the surface of the implant.

6. The method of claim 1, further comprising determining a quality of the bone using zones of the cross section of the bone.

7. The method of claim 6, wherein determining the quality of the bone includes determining a score for the quality of the bone in an anatomic location.

8. The method of claim 1, wherein a plurality of cross sections are oriented on the image along a longitudinal axis of the bone, and wherein bone quality is determined between a periphery and a central region of the cross section.

9. (canceled)

10. The method of claim 1, wherein the bone is a humerus and a joint that includes the bone is a shoulder.

11. A device for repairing a part of a bone in a subject, the device comprising:
a first section having a first material property;
a second section having a second material property;
the first section being connected to the second section at a central region of the device forming an asymmetric implant, wherein the first section and the second section are configured to reduce stress shielding to a region of the bone.

12. The device of claim 11, wherein the first material property includes at least one of elasticity, surface coating treatment, porosity, thickness, fin length, fin thickness, or implant shape.

13. The device of claim 11, wherein the second material property includes at least one of elasticity, surface coating treatment, porosity, thickness, fin length, fin thickness, or implant shape.

14. The device of claim 11, wherein at least one of the first material property or the second material property includes a surface coating treatment of the device, and wherein the surface coating treatment corresponds to a location of stress shielding of the bone.

15. The device of claim 14, wherein the surface coating treatment is asymmetric on the surface of the device.

16. The device of claim 14, wherein the location of stress shielding of the bone corresponds to a location of reduced bone thickness.

17. The device of claim 11, wherein at least one of the first material property or the second material property includes a fin length, and wherein the fin length is configured to engage with quality peripheral bone.

18. The device of claim 11, wherein at least one of the first material property or the second material property includes a fin thickness, and wherein the fin thickness is configured to engage with quality bone.

19. The device of claim 11, wherein the bone is a humerus and a joint that includes the bone is a shoulder.

20. A method for manufacturing an orthopedic implant for repairing a part of a bone in a subject, the method comprising:
forming the implant to include at least one material property determined by;
i) obtaining an image of the bone from at least one viewing plane;
ii) orienting on the image a cross section of the bone;
iii) quantifying a quality of the bone based on the cross section;
iv) determining the at least one material property of the implant to improve implant fixation to the bone using the quantified bone quality.

21. (canceled)

22. (canceled)

23. (canceled)

24. (canceled)

25. (canceled)

26. (canceled)

27. (canceled)

28. (canceled)

29. (canceled)

30. The device of claim 11:
wherein the first section and the second section are configured to improve implant fixation to a region of the bone.

31. (canceled)

32. (canceled)

33. (canceled)

34. (canceled)

35. (canceled)

36. (canceled)

37. (canceled)
